(19) United States
(12) Patent Application Publication
(10) Pub. No.: US 2005/0112684 A1
(43) Pub. Date: May 26, 2005

(54) Class I and Class II MHC Profiling for Social and Sexual Matching of Human Partners

(75) Inventor: Eric Holzle, Naples, FL (US)

Correspondence Address: Eric Holzle, 788 Park Shore Dr Ste F20, Naples, FL, 34103-3780, US

(73) Assignee: Eric Holzle, Naples, FL (US)

(21) Appl. No.: 10/707,124

(22) Filed: Nov. 21, 2003

(51) Int. Cl. G06F 17/60; G01N 33/53; G06F 19/00; G01N 33/48; G01N 33/50

(52) U.S. Cl. 435/7.1; 702/19; 705/3

(57) ABSTRACT

An improved process of matching people with one another, for social and/or sexual purposes, based on the profiles of the participants' Class I and Class II MHC genes. The quality of this human matching is inversely proportional to the number of common alleles of said genetic profiles of the individuals. Higher quality matches of this type result in greater sexual attraction, more attractive physical odor, and offspring with more robust immune systems among matched participants.

### BACKGROUND OF INVENTION

1. Field of Invention

This invention is a new use of known genetic phenomenon, and it relates to dating services and any other social or sexual matching of people. Specifically, it's the process of using partial genetic profiles to match individuals with partners.

2. Discussion of Prior Art

There are two general areas of prior art that relate to the invention. The first includes the fields from which the invention derives; those of zoology and reproductive genetics. The second category of relevant prior art is that to which the new use of the first is applied; namely dating services and partner-matching organizations in general.

Encouraged primarily by agricultural interests, zoologists have long studied sexual attraction of mammals, with an emphasis on how to promote it. In this context, pheromones were discovered and studied as chemicals excreted by many mammals to aid in the selection and attraction of potential mates. Recent studies show the correlation between individuals' genetic profiles and their pheromonic interactions with others.

Dating services and other partner-matching organizations have historically based their matches on many combinations of the participants' characteristics, without looking at their genetic profiles. The characteristics they've considered include those that are aesthetic, psychological, geographic, professional, and behavioral. Some dating services, for example, have distributed photographs of their members, amongst their other members, so that participants can select partners who appear attractive to them. Others have subjected participants to intelligence testing, in order to match partners of similar intellect. Still other services have rated partner compatibility on the preferred hobbies and activities of those considered.

The problem with all these approaches is that none of them, or any combination thereof, assure that the matched partners will experience a specific chemical compatibility with each other. A chemical mismatch of this type can result in a lack of sexual attraction between two individuals, an aversion to each other's natural body odor, and, if mating occurs, suboptimal immune systems in the resulting offspring.

Objects and Advantages: Accordingly, the primary object of this invention is the matching of potential social or sexual partners in such a way that enhances a specific genetic compatibility. This compatibility is based on the genes of the Major Histocompatibility Complex, or MHC, and its advantages over the existing art are numerous. They include increased sexual attraction, more appealing body odor, and healthier offspring among people matched in this way.

People matched together with MHC profiling are more desirable to each other sexually. This is because an individual's sexual chemicals, known as pheromones, are defined by MHC genes. Though odorless, pheromones attract potential mates, while repelling others. The new process described in this patent allows matches of people whose pheromones heighten their mutual sexual attraction.

Another advantage is that partners matched in this way have a more appealing natural body odor to each other. The better a couple is matched using MHC typing, the more attractive the two individuals find each other's natural scent. Likewise, a couple matched with clashing MHC profiles would find each others' body odor to be disagreeable. This new process can match couples with their best smelling counterparts. Or, it can be used to assure that partners matched with other processes aren't assigned with those whose natural scent is offensive to them.

Perhaps the most significant advantage of MHC profile matching is realized when two matched partners produce offspring. MHC profiling looks specifically at the genes that define the immune response in individuals. Offspring with the most robust immune systems are produced when their parents possess specific immune response genes that are different from each other. MHC typing, as described in this patent, allows potential parents to be matched so that together, they possess the highest diversity of those immune genes possible. All else being equal, this maximizes the quality of the immune systems of the offspring.

The benefits of MHC profiling when matching people in social and potentially sexual groups are numerous. Increased sexual attraction, appealing body odors, and healthier offspring are three factors that make this new process significantly improved over the prior art.

### SUMMARY OF INVENTION

This invention is the process of matching people, for sexual or social purposes, based on the compatibility of the Class I and Class II regions of their MHC genes.

### BRIEF DESCRIPTION OF DRAWINGS

FIG. 1 is a diagram of the overall process described in this patent. Specifically, it shows the process of using MHC profiling to match potential social or sexual partners together.

### BRIEF DESCRIPTION OF SEQUENCES

There are no new DNA sequences introduced in this patent. However, known genes are analyzed in this new process. Specifically, this invention examines the sequences of genes in the Class I and Class II regions of the Major Histocompatibility Complex, known as the MHC genes. They're located on the short arm of chromosome 6 (6p). The Class I region includes the gene groups HLA-A, -B, -C, and -G. The Class II region contains the gene groups HLA-DPA, -DPB, -DQA, -DQB, -DRA, -DRB, -DNA, -DOB, -LMP, and -TAP.

### DETAILED DESCRIPTION

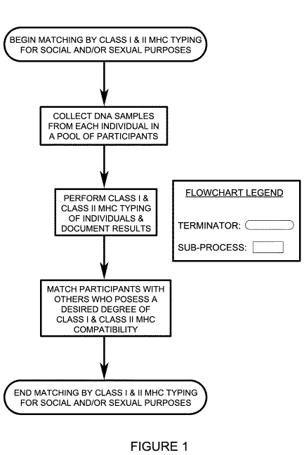

As FIG. 1 shows, the process of matching people using MHC profiling can be broken down into three basic steps: Collecting genetic samples from the individuals in a pool of participants, typing the Class I and Class II regions of the MHC of each individual, and matching together those individuals with complimentary Class I and Class II MHC profiles. The first two steps are routinely practiced today in the field of genetics, and the third completes the process of this invention.

Collecting sample genetic material from the individuals in a pool of participants is relatively common. It could include drawing blood, rubbing a cotton swab along the inside of the cheek to retrieve cells from the mouth, or removing hair. Samples are often labeled and documented to associate them with the individuals from whom they came.

Once genetic material is obtained, it can be analyzed, or typed, to determine the individuals' MHC Class I and Class II profiles. MHC typing is commonly practiced today in genetic laboratories, and it often involves growing the original DNA sample to establish a desired quantity, followed by the actual typing itself. Both of these steps are accomplished in a variety of ways, which are outside the scope of this patent, but the end result is a Class I and Class II MHC profile of each subject. The results of this MHC typing are often documented for reference.

The concluding step in this new process is the actual matching of individuals based on the Class I and Class II profiles of their MHC. The most compatible human matches occur when the participants have no common alleles in the Class I and Class II regions of their MHC genes. The degree of compatibility is inversely proportional to the number of alleles individuals have in common with each other. The least compatible individuals have all the same alleles in the Class I and Class II regions of their MHC genes.

Collectively, the three basic steps of gathering genetic samples, analyzing those samples to determine the Class I and Class II profiles of the MHC genes of each participant, and matching the participants based on those genetic profiles, represent the overall process described in this patent.

1. A method of matching human beings with others, comprising the steps of:
(a) assembling and/or defining a population of human participants, physically and/or virtually, to be matched amongst themselves and/or any future or past participants in the context of a dating service, dating services, or other social groups or organizations;
(b) producing, assembling, and/or observing the class I and class II MHC profiles, in any part or in whole, of all or any fraction of the participants;
(c) comparing said profiles of some or all of the participants with said profiles of others and rating the degree of compatibility between any two or more people according to the number of alleles they have in common, where fewer commonalities represent a greater degree of compatibility;
(d) matching said participants based on said comparisons.

2. A method of matching human beings with others, comprising the steps of:
(a) assembling and/or defining a population of human participants, physically and/or virtually, to be matched amongst themselves and/or any future or past participants in the context of a dating service, dating services, or other social groups or organizations;
(b) producing, assembling, and/or observing the class I and class II MHC profiles, in any part or in whole, of all or any fraction of the participants, where said profiles include the HLA-A and HLA-B loci in the class I region, and the DRB1 locus in the class 11 region;
(c) comparing said profiles of some or all of the participants with said profiles of others and rating the degree of compatibility between any two or more people according to the number of alleles they have in common, where fewer commonalities represent a greater degree of compatibility;
(d) matching said participants based on said comparisons.

3. A method of matching human beings with others, comprising the steps of:
(a) assembling and/or defining a population of human participants, physically and/or virtually, to be matched amongst themselves and/or any future or past participants in the context of a dating service, dating services, or other social groups or organizations;
(b) producing, assembling, and/or observing the class I and class II MHC profiles of all or any fraction of the participants, where said profiles include the HLA-A and HLA-B loci in the class I region, and the DRB1 locus in the class II region;
(c) comparing said profiles of some or all of the participants with said profiles of others and rating the degree of compatibility between any two or more people according to the number of alleles they have in common, where fewer commonalities represent a greater degree of compatibility;
(d) matching said participants based on said comparisons.
